(19) United States
(12) Patent Application Publication
(10) Pub. No.: US 2017/0055884 A1
(43) Pub. Date: Mar. 2, 2017

(54) MOTOR FUNCTION EVALUATION SYSTEM, MOTOR FUNCTION EVALUATION METHOD, MOTOR FUNCTION EVALUATION PROGRAM, AND EVALUATION DEVICE

(71) Applicant: SEIKO EPSON CORPORATION, Tokyo (JP)

(72) Inventor: Jun TAKEUCHI, Matsumoto-shi (JP)

(73) Assignee: SEIKO EPSON CORPORATION, Tokyo (JP)

(21) Appl. No.: 15/249,396

(22) Filed: Aug. 27, 2016

(30) Foreign Application Priority Data
Aug. 27, 2015 (JP) 2015-168110

(51) Int. Cl. A61B 5/11; A61B 5/103; A47K 17/02; A61B 5/00

(52) U.S. Cl. CPC A61B 5/1124; A61B 5/702; A61B 5/1036; A61B 5/7221; A61B 5/486; A47K 17/028; A61B 2560/0475; E03D 11/02

(57) ABSTRACT

A motor function evaluation system includes: a height changing unit (lift mechanism) which changes a relative height dimension between a seat surface where a user sits and a placement surface where the user's soles are placed; a first pressure detection unit which detects a pressure applied by the soles; a movement details reporting unit (reporting unit) which reports details of a motion test that should be carried out by the user; and an evaluation unit which evaluates a motor function of the user on the basis of a detection result by the first pressure detection unit when the motion test is carried out by the user.

This application claims the benefit of Japanese Patent Application No. 2015-168110, filed on Aug. 27, 2015. The content of aforementioned application is incorporated herein by reference in its entirety.

### BACKGROUND

1. Technical Field

The present invention relates to a motor function evaluation system, a motor function evaluation method, a motor function evaluation program, and an evaluation device.

2. Related Art

According to the related art, an exercise prescription system is known in which an exercise to be suggested to a subject is prescribed according to the result of the determination of the physical strength of the subject (see JP-A-2013-252418, for example).

The exercise prescription system disclosed in JP-A-2013-252418 is made up of an exercise prescription device and an ultrasonic measuring device and is used in the case of recommending an exercise menu suitable for the subject in a fitness gym or the like, for example.

Of these devices, the ultrasonic measuring device is brought in tight contact with a body site of the subject and thus outputs image data representing an echo image of the inside of the body site. The exercise prescription device is connected to the ultrasonic measuring device and selects and generates prescription information on the basis of a muscle thickness evaluation value and a fat thickness evaluation value indicating the evaluation of muscle thickness and fat thickness that are inputted respectively, and a motor ability evaluation value (indicator of the evaluation of motor ability) that is inputted. The prescription information is text information showing details of an exercise that should be carried out by the subject. The prescription information is outputted on a display when a viewing instruction is inputted by the subject. By employing the motor ability evaluation value as an element in selecting such prescription information, it is possible to offer the elderly appropriate advice on exercise to prevent locomotive syndrome.

Locomotive syndrome is a term proposed by the Japan Orthopaedic Association and expressing the state where the mobility function has fallen because of a disorder of motor organs such as muscles, bones, joints, cartilage and intervertebral discs.

Incidentally, if locomotive syndrome advances, the person has trouble with everyday living. Therefore, it is necessary to keep motor organs in good condition in daily living, thus prevent the state of being diagnosed with locomotive syndrome, and extend health life expectancy.

However, the exercise prescription system disclosed in JP-A-2013-252418 is a system used in a fitness gym or the like. Similarly, while the “locomotive syndrome risk test” or the like for checking the mobility function is proposed by the Japan Orthopaedic Association, it is necessary to carry out the test deliberately at a medical institution or the like in order to get it done correctly. Therefore, there is a problem that, for ordinary people including the elderly who tend to have a disorder of motor organs, it is difficult to carry out the test easily in everyday living and that it is also difficult to continuously observe changes in the mobility function. Based on such a problem, a configuration and method that enables ordinary people to easily measure the state of their own motor organs in everyday living has been demanded.

### SUMMARY

An advantage of some aspects of the invention is to provide a motor function evaluation system, a motor function evaluation method, a motor function evaluation program and an evaluation device that enable easy evaluation of the motor function of a user.

A motor function evaluation system according to a first aspect of the invention includes: a height changing unit which changes a relative height dimension between a seat surface where a user sits and a placement surface where the user's soles are placed; a first pressure detection unit which detects a pressure applied by the soles; a movement details reporting unit which reports details of a motion test that should be carried out by the user; and an evaluation unit which evaluates a motor function of the user on the basis of a detection result by the first pressure detection unit when the motion test is carried out by the user.

As the details of the motion test, a locomotive syndrome determination test can be given as an example. In such a determination test, the user stands up on one foot or on both feet from the state of being seated on platforms with different heights, and whether the user can maintain the stand-up state for a predetermined period of time or not is determined. Then, in this determination test, the motor function of the user is evaluated on the basis of the height dimension of the lowest platform, of the platforms from which the user successfully stands up.

According to the first aspect, the relative height dimension between the seat surface where the user sits and the placement surface where the user's soles are placed is adjusted. Then, details of a motion test are reported, and the motor function of the user is evaluated on the basis of the result of the detection of the pressure applied by the soles when the motion test is carried out. This enables the motion test to be easily carried out by providing the foregoing configuration in a high chair or toilet which is used frequently in everyday living. Therefore, the motor function of the user can be evaluated relatively easily.

In the first aspect, it is preferable that the motor function evaluation system includes a result reporting unit which reports an evaluation result of the motor function by the evaluation unit.

According to this configuration, the user or other people such as medical practitioners can grasp the motor function of the user by checking the evaluation result reported by the result reporting unit. Thus, measures corresponding to the motor function of the user can be taken.

In the first aspect, it is preferable that the result reporting unit reports a comparison result between a motor function level based on the detection result by the first pressure detection unit and the motor function level that is an average among at least either those of the same age group as the user or those of the same gender as the user.

According to this configuration, the user is enabled to easily grasp whether the user's own motor function is superior or inferior to at least those of the same age group or those of the same gender by confirming the reported comparison result.

In the first aspect, it is preferable that the height changing unit is configured to be able to change the relative height dimension by changing a height position of the placement surface.

Here, in the case of adjusting the height position of the seat surface, it is conceivable that the height position of the structure including the seat surface is to be adjusted. However, if this structure is provided with wiring and piping (for example, a toilet bowl), the configuration and design become complicated by needing changes to the wiring and piping, or the like.

In contrast, according to the foregoing configuration, the height dimension can be changed simply by moving up and down the area where the user's soles are placed on the floor or the like. Thus, the configuration of the height changing unit can be simplified.

In the first aspect, it is preferable that the motor function evaluation system includes a success/failure determination unit which determines a success/failure of the motion test on the basis of a change in gravity center position of the user acquired from the detection result by the first pressure detection unit, and that the evaluation unit evaluates the motor function of the user on the basis of a determination result by the success/failure determination unit.

According to this configuration, the success/failure determination unit can properly determine the success/failure of the motion test. Also, since the evaluation unit evaluates the motor function of the user on the basis of the determination result by the success/failure determination unit, the motor function can be properly evaluated.

In the first aspect, it is preferable that the motor function evaluation system includes a second pressure detection unit which detects the user's pressure to the seat surface, and a notice reporting unit which reports a notice to the user, and that the success/failure determination unit determines whether a movement carried out by the user is a movement using a bounce or not, on the basis of a detection result by the second pressure detection unit, and causes the notice reporting unit to report a notice if the movement is determined as a movement using a bounce.

Here, in the determination test for locomotive syndrome, standing up by using a bounce is not regarded as a success. Therefore, if such a determination test is included in the motion test, the success/failure determination unit determines whether the movement of the user is made by using a bounce or not, and thus the success/failure of the motion test can be properly determined. Also, if the movement is determined as using a bounce, the notice reporting unit reports a notice. Therefore, the user carrying out the motion test can carry out the motion test again, paying attention not to use a bounce. Thus, the user can be made to carryout the motion test properly.

In the first aspect, it is preferable that the motor function evaluation system includes a storage unit which stores the evaluation result by the evaluation unit, and a transition reporting unit which reports a transition of the evaluation result by the evaluation unit.

According to this configuration, the transition reporting unit reports the transition of each of evaluation results stored in the storage unit. Therefore, the user can reliably grasp changes in the user's own motor function by checking the transition.

In the first aspect, it is preferable that the motor function evaluation system includes a transition prediction unit which predicts a future transition of the motor function of the user on the basis of the transition of the evaluation result by the evaluation unit, and a prediction result reporting unit which reports a prediction result by the transition prediction unit.

According to this configuration, a predicted future transition of the motor function is reported. Therefore, if this transition is not good, the user can be given a sense of urgency and prompted to make improvements. Meanwhile, if this transition is good, the user can be given a sense of relief.

In the first aspect, it is preferable that the motor function evaluation system includes an exercise reporting unit which reports details of an exercise that should be carried out by the user, on the basis of the evaluation result by the evaluation unit.

According to this configuration, details of an exercise suitable for the user can be reported. By carrying out this exercise, the user can restrain a reduction in the motor function or can improve the motor function.

In the first aspect, it is preferable that the motor function evaluation system includes a change estimation unit which estimates a change in the motor function in the case where the user carries out the exercise, and an estimated state reporting unit which reports the estimated change in the motor function of the user.

According to this configuration, a change in the motor function of the user that is estimated in the case where the exercise suitable for the user is carried out is reported. Thus, the user can be motivated to carry out the exercise and can be prompted to carry out the exercise.

In the first aspect, it is preferable that the motor function evaluation system includes a transmitting unit which transmits information based on the evaluation result by the evaluation unit to an external device.

According to this configuration, the determination result can be accumulated in an external device. Therefore, the determination result can be statistically processed by the external device, for example. Moreover, since the external device acquires the determination result, the user can check the determination result even when the user is not in the place where the height changing unit is situated, and therefore the convenience of the motor function evaluation system can be improved.

A motor function evaluation method according to a second aspect of the invention includes: adjusting a relative height dimension between a seat surface where a user sits and a placement surface where the user's soles are placed; reporting details of a motion test that should be carried out by the user; and evaluating a motor function of the user on the basis of a change in a pressure applied to the placement surface by the user's soles when the motion test is carried out by the user.

According to the second aspect, effects similar to those of the motor function evaluation system according to the first aspect can be achieved.

A motor function evaluation program according to a third aspect of the invention is executed by an evaluation device which evaluates a motor function of a user and causes the evaluation device to execute: causing a height changing device which changes a relative height dimension between a seat surface where a user sits and a placement surface where the user's soles are placed, to adjust the height dimension; causing a reporting device to report details of a motion test that should be carried out by the user; and evaluating a motor function of the user on the basis of a detection result by a pressure detection device which detects a change in a pressure applied to the placement surface by the user's soles when the motion test is carried out by the user.

According to the third aspect, as the evaluation device executes the motor function evaluation program, the evaluation device can evaluate the motor function of the user relatively easily, similarly to the evaluation unit in the motor function evaluation system according to the first aspect.

An evaluation device according to a fourth aspect of the invention includes: a height acquisition unit which acquires a relative height dimension between a seat surface where a user sits and a placement surface where the user's soles are placed; a change acquisition unit which acquires a change in a pressure applied to the placement surface by the user's soles when a motion test is carried out by the user; and an evaluation unit which evaluates a motor function of the user on the basis of the height dimension and the change in the pressure.

According to the fourth aspect, the motor function of the user can be evaluated relatively easily, as in the motor function evaluation system according to the first aspect.

A motor function evaluation program according to a fifth aspect of the invention is executed by an evaluation device which evaluates a motor function of a user and causes the evaluation device to execute: acquiring a relative height dimension between a seat surface where a user sits and a placement surface where the user's soles are placed; acquiring a change in a pressure applied to the placement surface by the user's soles when a motion test is carried out by the user; and evaluating the motor function of the user on the basis of the height dimension and the change in the pressure.

According to the fifth aspect, as the evaluation device executes the motor function evaluation program, the evaluation device can evaluate the motor function of the user relatively easily, similarly to the evaluation unit in the motor function evaluation system according to the first aspect.

### BRIEF DESCRIPTION OF THE DRAWINGS

The invention will be described with reference to the accompanying drawings, wherein like numbers reference like elements.

FIG. 1 is a schematic view showing the configuration of a motor function evaluation system according to an embodiment of the invention.

FIG. 2 is a schematic view showing details of a motion test carried out by a user in the embodiment.

FIG. 3 is a side view showing the arrangement positions of a detection device, a lift device and an operation panel in the embodiment.

FIG. 4 is a plan view showing the arrangement positions of the detection device and the lift device in the embodiment.

FIG. 5 is a block diagram showing the configuration of a control device in the embodiment.

FIG. 6 is a side view showing the state where a motion test is carried out in the embodiment.

FIG. 7 is a side view showing the state where a motion test is carried out in the embodiment.

FIG. 8 is a block diagram showing the configuration of a server in the embodiment.

FIG. 9 is a flowchart showing motor function evaluation processing in the embodiment.

FIG. 10 shows an example of an evaluation result screen in the embodiment.

### DESCRIPTION OF EXEMPLARY EMBODIMENTS

Hereinafter, an embodiment of the invention will be described with reference to the drawings.

### Configuration of Motor Function Evaluation System

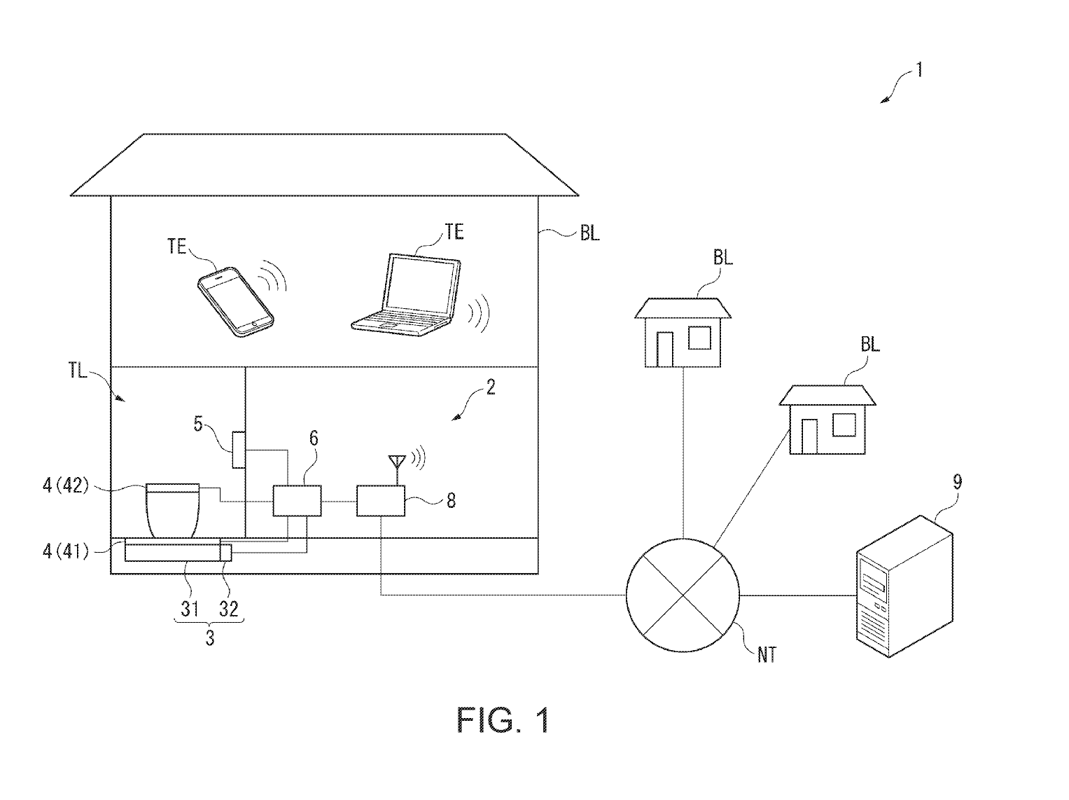

FIG. 1 is a schematic view showing the configuration of a motor function evaluation system 1 according to this embodiment.

The motor function evaluation system 1 according to this embodiment includes a motor function evaluation device 2 provided inside a building BL such as a house or nursing facility where a user leads his/her everyday life, and a server connected for communication with the motor function evaluation device 2 via a network NT such as the Internet, and also includes an information processing device TE according to need, as shown in FIG. 1. The motor function evaluation system 1 is mainly targeted at the elderly as the user and is configured to cause the user to carry out a predetermined motion test in a toilet TL inside the building BL and thus determine the motor function of the user (mobility function as described above, for example) and present the result of the determination or the like.

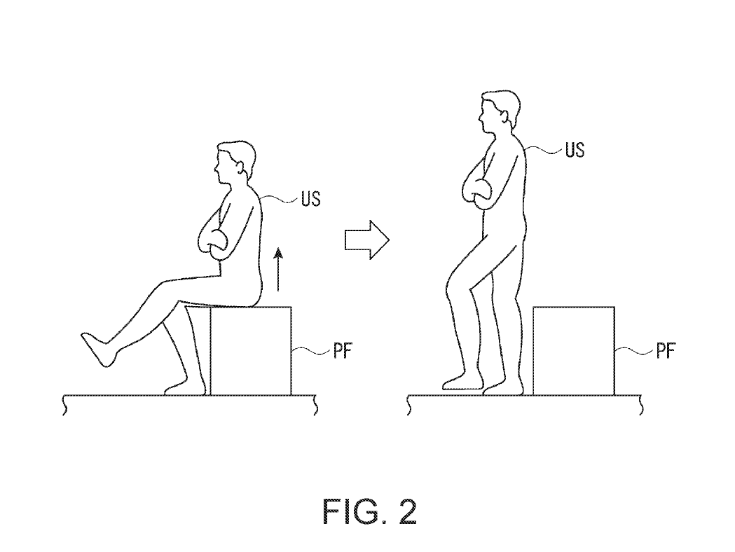

FIG. 2 is a schematic view showing details of a motion test carried out by the user.

Here, the motion test carried out by the user in the embodiment is a motion test similar to a determination test for determining the user's stage of locomotive syndrome shown in FIG. 2.

Hereinafter, the determination test for locomotive syndrome similar to the motion test in the embodiment will be described.

In this determination test, the user stands upright without using a bounce from the state of being seated on a platform with a height of 40 cm, 30 cm, 20 cm or 10 cm, and the success/failure is determined on the basis of whether the user can maintain the stand-up state for three seconds or not.

Specifically, in this determination test, first, a user US stands up on one foot from the state of being seated on a platform PF with a height of 40 cm, and maintains the stand-up state for three seconds. If the user successfully maintains the stand-up state on one foot with respect to both the left and right feet, the user then stands up on one foot with respect to both the left and right feet from the state of being seated on a platform that is 10-cm lower, and maintains the stand-up state for three seconds. Then, the lowest platform from which the user successfully stands up and maintains the stand-up state on one foot for three seconds with respect to both the left and right feet is the test result.

Meanwhile, if the user has failed in the determination test with one foot using the platform with a height of 40 cm, the user then stands up on both feet. If the user successfully stands up on both feet, the user then stands up on both feet from the state of being seated on a platform that is 10-cm lower. Then, the lowest platform from which the user successfully stands up and maintains the stand-up state on both feet for three seconds is the test result.

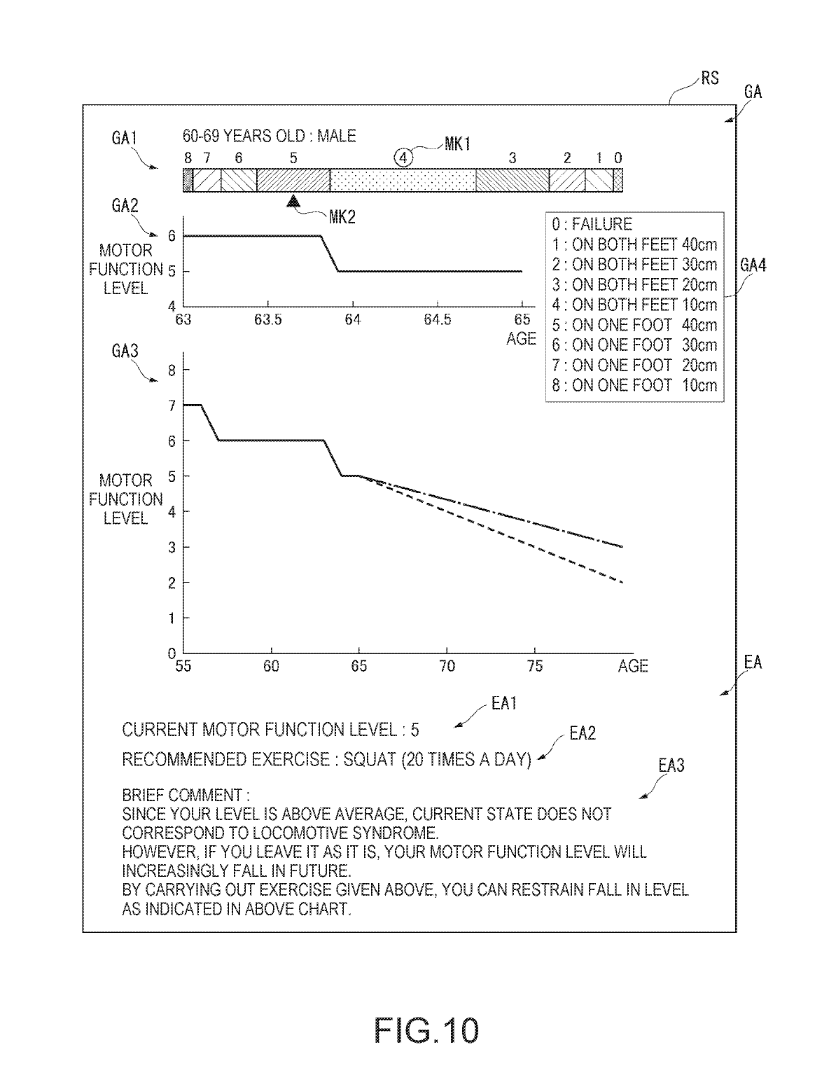

The motor function evaluation system 1 (motor function evaluation device 2) according to the embodiment enables the user to carry out a motion test similar to the above determination test, using a toilet bowl which the user uses in everyday living, as the platform. The motor function evaluation system 1 determines and records the motor function level based on the test result of the user and also enables reporting of an evaluation result screen RS (see FIG. 10) based on the test result, or the like.

The configuration of each part in the motor function evaluation system 1 will be described in detail below.

### Configuration of Motor Function Evaluation Device

The motor function evaluation device 2 is configured to cause a user to carry out a motion test and to evaluate the motor function of the user and present the result of the evaluation on the basis of a movement of the user that is detected. This motor function evaluation device 2 includes a lift mechanism 3, a detection device 4, an operation panel 5, a control device 6, and a relay device 8, as shown in FIG. 1.

Of these, the lift mechanism 3, the detection device 4, and the operation panel 5 are arranged in the toilet TL inside the building BL. Meanwhile, the control device 6 and the relay device 8 need not necessarily be provided in the toilet TL, as long as the control device 6 is arranged at a position where the control device 6 can communicate with the lift mechanism 3, the detection device 4, and the operation panel 5, and as long as the relay device 8 is arranged at a position where the relay device 8 can communicate with the control device 6.

### Configuration of Lift Mechanism

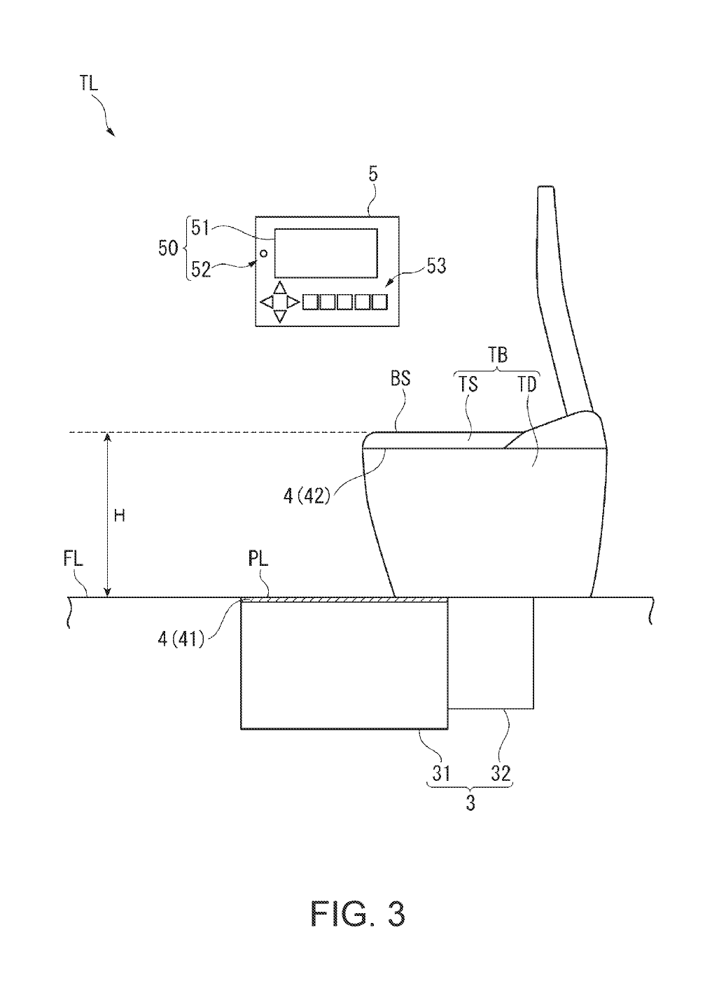

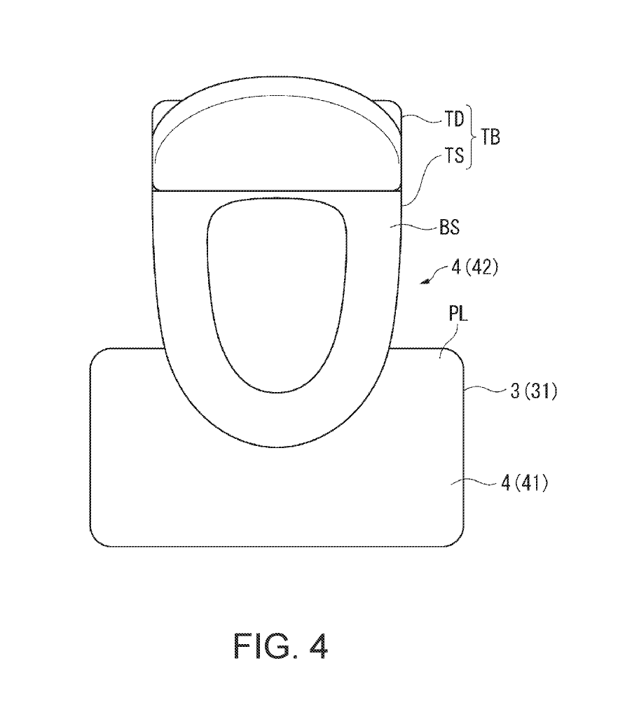

FIG. 3 is a side view showing the arrangement positions of the lift mechanism 3, the detection device 4, and the operation panel 5 in the toilet TL. FIG. 4 is a plan view showing the arrangement positions of the lift mechanism 3 and the detection device 4.

The lift mechanism 3 forms the height changing unit and the height changing device according to the invention. The lift mechanism 3 changes a relative height dimension H between a placement surface PL where both feet of the user (soles of both feet) are placed when the user sits on a seat surface BS of a toilet bowl TB, in a floor FL of a toilet TL, and the seat surface BS of the toilet bowl TB arranged inside the toilet TL, as shown in FIGS. 3 and 4. Specifically, the lift mechanism 3 has a moving unit 31 including the placement surface PL, and a lift device 32 which moves the moving unit 31 up and down.

The placement surface PL of the moving unit 31, where the soles are placed, is flush with the floor FL in the state where the moving unit 31 is not moved, as shown in FIG. 3.

The lift device 32 has a motor, a plurality of gears rotated by the driving of the motor, and a guide rail for guiding the moving unit 31 to move up and down, though not illustrated. The lift device 32 moves the moving unit 31 up and down at a 10-cm interval on the basis of a control signal inputted from the control device 6. However, the interval at which the lift device 32 moves the moving unit 31 up and down can be changed suitably and may be 1 cm or 5 cm.

Also, the lift device 32 moves the moving unit 31 up and down within such a range that the placement surface PL is not above the seat surface BS. More specifically, the lift device 32 moves the moving unit 31 up and down to such height positions that the height dimension H falls within a range of 10 cm or above and 40 cm or below. However, this is not limiting. The height position of the moving unit 31 moved up and down by the lift device 32 can be changed suitably.

### Configuration of Detection Device

The detection device 4 includes a first pressure detection unit 41 and a second pressure detection unit 42, as shown in FIGS. 3 and 4.

The first pressure detection unit 41 is a pressure sensor arranged in the placement surface PL. That is, the first pressure detection unit 41 is provided in the moving unit 31. The first pressure detection unit 41 detects a pressure applied by the user's soles.

The second pressure detection unit 42 is a pressure sensor which detects a pressing force applied to a toilet seat TS. The second pressure detection unit 42 is provided either in the toilet seat TS or in a toilet bowl main body TD on which the toilet seat TS is provided. For example, the second pressure detection unit 42 is provided on the seat surface BS or on a surface of one of the toilet seat TS and the toilet bowl main body TD that is opposite the other.

### Configuration of Operation Panel

The operation panel 5 has a display section 51, an audio output section 52, and an operation section 53. Of these, the display section 51 and the audio output section 52 form a reporting unit 50. The reporting unit 50 forms the reporting device according to the invention.

The display section 51 is made up of various displays such as a liquid crystal display and displays an image corresponding to an image signal inputted from the control device 6.

The audio output section 52 has a speaker and outputs a sound corresponding to an audio signal inputted from the control device 6.

The operation section 53 has various buttons and outputs an operation signal corresponding to the pressing of the buttons to the control device 6. However, the operation section 53 is not limited to such a configuration with buttons and may have a configuration with a touch panel covering the display surface of the display section 51, instead of or in addition to these buttons.

### Configuration of Control Device

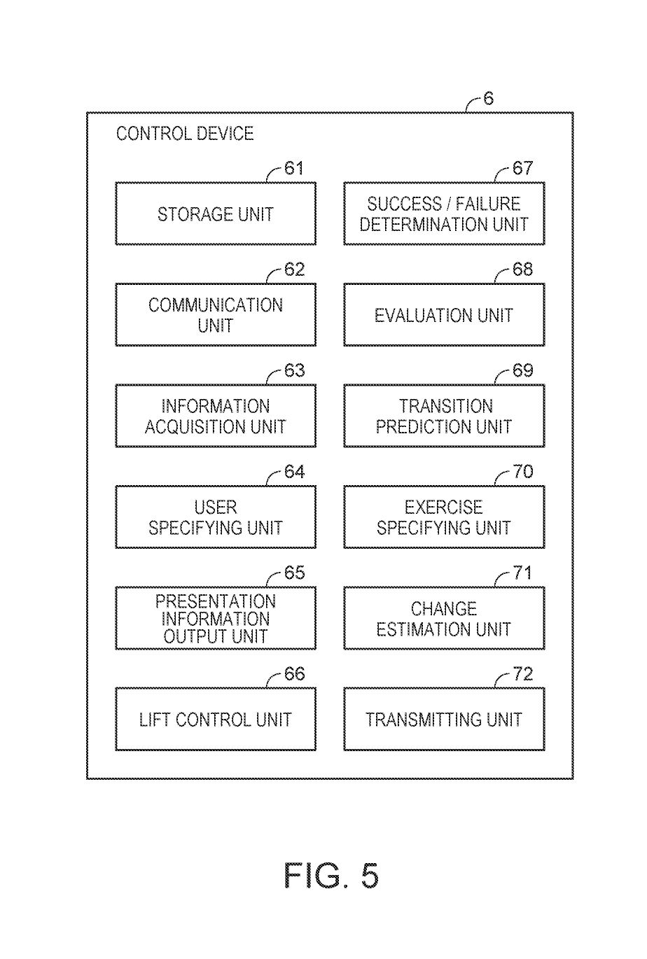

FIG. 5 is a block diagram showing the configuration of the control device 6.

The control device 6 has a circuit board on which a processing circuit such as a CPU (central processing unit) and a storage circuit such as a flash memory are installed. The control device 6 is connected to the lift mechanism 3, the detection device 4, and the operation panel 5, and controls the operation of these components and also communicates with the server 9 and the information processing device TE, described later, via the relay device 8. Therefore, the control device 6 has a storage unit 61 made up of the storage circuit, and a communication unit 62, as shown in FIG. 5.

The control device 6 also causes the reporting unit 50 to output a predetermined image and sound and causes the user to carryout the motion test. Moreover, the control device 6 evaluates the motor function of the user on the basis of the detection result by the detection device 4 when the motion test is carried out. In addition, the control device 6 stores user information inputted using the operation section 53, and evaluation result information including the evaluation result of the motor function, and transmits these items of information to the server 9.

The user information is information including personal information of the user and may be user ID, name, gender, age, and body weight. Of these, the user ID is an ID allocated to each user who uses the motor function evaluation device 2 in each building BL.

The evaluation result information is information indicating the evaluation result of the motor function of the user based on the test result of the motion test, and includes user ID, building ID, time and date when the motion test is carried out, and motor function level or the like, described later. Of these, the building ID is an ID allocated to the building BL used by each user.

To realize such functions, the control device 6 has an information acquisition unit 63, a user specifying unit 64, a presentation information output unit 65, a lift control unit 66, a success/failure determination unit 67, an evaluation unit 68, a transition prediction unit 69, an exercise specifying unit 70, a change estimation unit 71, and a transmitting unit 72, which respectively function by the CPU performing the program stored in the storage unit 61. That is, the control device 6 is equivalent to an evaluation device which executes a motor function evaluation program and thus evaluates the motor function of the user.

### Configuration of Storage Unit

The storage unit 61 stores a program and data that are necessary for controlling the motor function evaluation device 2. For example, as the program, the storage unit 61 stores a motor function evaluation program which causes motor function evaluation processing, later described, to be executed. As the data, the storage unit 61 stores the user information, the detection result, and the evaluation result, and also stores statistical data for each age group and each gender. Moreover, the storage unit 61 stores an image (including dynamic image) and sound indicating a method for carrying out the motion test, and type of exercise corresponding to the motor function level, described later.

### Configuration of Communication Unit

The communication unit 62 is an input/output interface connected to the components 3 to 5 and 8. To the communication unit 62, detection signals indicating the detection results by the respective detection units 41, 42 are inputted from the detection device 4, and an operation signal is inputted from the operation section 53. Also, the communication unit 62 outputs an image signal and an audio signal outputted from the presentation information output unit 65, described later, to the reporting unit 50, and outputs a control signal outputted from the lift control unit 66, described later, to the lift device 32 of the lift mechanism 3. Moreover, the communication unit 62 transmits and receives information to and from the relay device 8.

### Configuration of Information Acquisition Unit

The information acquisition unit 63 acquires various kinds of information. Specifically, the information acquisition unit 63 acquires the content of an operation by the user on the basis of an operation signal inputted from the operation section 53, and acquires information based on various signals acquired via the communication unit 62. For example, on the basis of a detection signal and an operation signal inputted to the communication unit 62, the information acquisition unit 63 acquires the detection result and the operation content indicated by these signals. The information acquisition unit 63 also acquires the height dimension H adjusted by the lift mechanism 3. That is, the information acquisition unit 63 has the functions of the height acquisition unit and the change acquisition unit. Moreover, the information acquisition unit 63 acquires information received by the relay device 8 (for example, information transmitted from the server 9 and the information processing device TE) via the communication unit 62.

### Configuration of User Specifying Unit

The user specifying unit 64 specifies a user (executioner) of an operation for carrying out the motion test (operation for executing motor function evaluation processing) when an operation signal corresponding to this operation is inputted from the operation section 53. In the embodiment, when the executioner carries out an operation of selecting him/herself from preregistered users, the user specifying unit 64 specifies this executioner.

However, the user specifying unit 64 may specify the executioner by another method. For example, the user specifying unit 64 may specify the executioner on the basis of the inputted user ID or may specify the executioner, using the recognition of fingerprints, vein patterns, retinal patterns, and voiceprints or the like, or face recognition. Moreover, the user specifying unit 64 may specify the executioner on the basis of the detection result when the executioner gets on the first pressure detection unit 41.

### Configuration of Presentation Information Output Unit

The presentation information output unit 65 is a functional unit which executes the functions of the movement details reporting unit, the notice reporting unit, the result reporting unit, the transition reporting unit, the prediction result reporting unit, the exercise reporting unit, and the estimated state reporting unit according to the invention.

The presentation information output unit 65 generates presentation information to be presented to the user and outputs signals (image signal and audio signal) corresponding to the presentation information to the reporting unit 50 via the communication unit 62. Thus, the reporting unit 50 reports the presentation information.

Specifically, the presentation information output unit 65 functions as the movement details reporting unit when an operation signal to the effect that the motion test is to be carried out is inputted from the operation section 53. In this case, the presentation information output unit 65 reads out image information and audio information indicating a method for carrying out the motion test, from the storage unit 61, and outputs an image signal and an audio signal corresponding to these items of information.

The presentation information output unit 65 functions as the notice reporting unit when it is determined by the success/failure determination unit 67, later described, that the user stands up using a bounce in the motion test. In this case, the presentation information output unit 65 outputs a message indicating a notice “Do not use a bounce”, and causes the reporting unit 50 to report this message.

Moreover, the presentation information output unit 65 functions as the result reporting unit, the transition reporting unit, the prediction result reporting unit, the exercise reporting unit, and the estimated state reporting unit, when the motion test, described later, is finished. In this case, the presentation information output unit 65 generates an evaluation result screen RS (see FIG. 10) including an evaluation result by the evaluation unit 68, a transition of the past evaluation result, a prediction result of the future motor function, a type of exercise that should be carried out by the user, and a change in the motor function in the case where this exercise is carried out. These details of the evaluation result screen RS are based on processing results by the functional units 68 to 71, described later.

As an image signal of the evaluation result screen RS is outputted to the reporting unit 50, this evaluation result screen RS is displayed in the display section 51. The details of the evaluation result screen RS will be described later.

### Configuration of Lift Control Unit

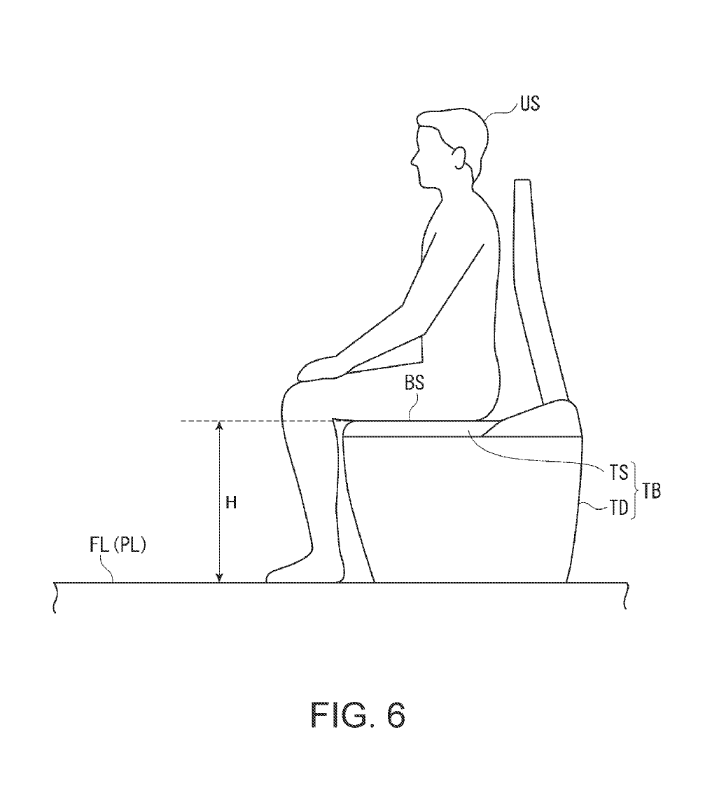

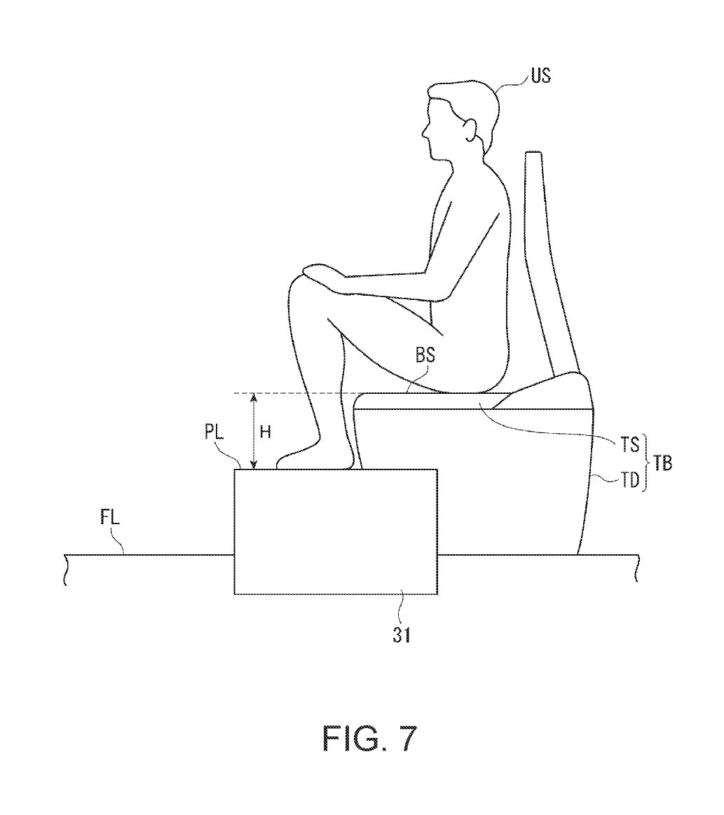

FIGS. 6 and 7 are side views showing the state where the motion test is carried out. Specifically, FIG. 6 shows the state where the height dimension H from the placement surface PL to the seat surface BS is 40 cm. FIG. 7 shows the state where the height dimension H is 20 cm.

The lift control unit 66 functions when the user US carries out the motion test. The lift control unit 66 controls the operation of the lift device 32 so as to adjust the amount of protrusion of the moving unit 31 from the floor FL and thus adjust the height dimension H from the placement surface PL to the seat surface BS.

Thus, the position of the placement surface PL can be adjusted from the state where the height dimension H from the placement surface PL to the seat surface BS is 40 cm (where the moving unit 31 is not moved up) as shown in FIG. 6, to the state where the height dimension H is reduced as shown in FIG. 7. Then, in each state, the motion test is carried out by the user US according to the details of the motion test reported by the reporting unit 50.

### Configuration of Success/Failure Determination Unit

Back to FIG. 5, the success/failure determination unit 67 functions at the time of carrying out the motion test and determines the success/failure of the motion test carried out by the user, on the basis of the detection result acquired by the information acquisition unit 63.

Specifically, the success/failure determination unit 67 determines whether the motion test in which the relative height dimension H between the placement surface PL and the seat surface BS is a predetermined value is successful or not, on the basis of changes with time in each of the load and the gravity center position corresponding to the detection result by the first pressure detection unit 41 at the time of the motion test.

The success/failure determination unit 67 also functions as a bounce determination unit if it is determined that the motion test is successful, on the basis of the detection result by the first pressure detection unit 41, and determines whether the user uses a bounce or not, on the basis of the detection result by the second pressure detection unit 42 at the time of the motion test.

If it is determined that the user uses a bounce, the success/failure determination unit 67 determines that this motion test is not successful. In this case, an image signal and an audio signal indicating “Do not use a bounce” are outputted by the presentation information output unit 65, as described above.

Meanwhile, the success/failure determination unit 67 determines that the motion test is successful if it is determined that the user succeeds without using a bounce. In this case, an image and a sound indicating a method for carrying out the next motion test are reported by the presentation information output unit 65 and the reporting unit 50.

For example, as described with respect to the procedures of the motion test, if it is determined that the user succeeds in the motion test on one of the right and left feet, the reporting unit 50 reports an image and a sound causing the user to carry out the motion test on the other foot. If it is determined that the user succeeds in the motion test on the left foot and on the right foot in the state where the height dimension from the placement surface PL to the seat surface BS is the same, the reporting unit 50 then reports details of the next motion test. In this case, the moving unit 31 is moved up by the lift control unit 66 in such a way as to reduce the height dimension H by 10 cm.

Meanwhile, if it is determined by the success/failure determination unit 67 that the user does not succeed in the motion test on one foot, the reporting unit 50 reports an image and a sound causing the user to carry out the motion test on both feet. Then, if it is determined that the user succeeds in the motion test on both feet, the reporting unit 50 reports details of the next motion test. In this case, similarly to the above, the moving unit 31 is moved up by the lift control unit 66 in such a way as to reduce the height dimension H by 10 cm.

However, if it is determined that the user fails in each of the motion test on the left foot and on the right foot and the motion test on both feet, and if it is determined that the user succeeds in the motion test on the left foot and on the right foot or on both feet with the height dimension H of 10 cm, the motion test is finished. In this case, the reporting unit 50 reports that the motion test is finished, and the lift control unit 66 returns the height position of the placement surface PL to substantially the same position as the floor FL.

As each motion test is thus finished, the success/failure determination unit 67 regards the details of the motion test with the smallest height dimension H of the motion tests in which the user succeeds on the left foot and on the right foot or on both feet, as the test result. For example, if the motion test with the smallest height dimension H of the motion tests in which the user succeeds is the motion test on the left foot and on the right foot with the height dimension H of 20 cm, details to that effect form the test result for the user.

If the user does not succeed in the motion test on both feet with the height dimension H of 40 cm, details to the effect that the user does not succeed in any motion test form the test result for the user. The success/failure determination unit 67 stores the test result in the storage unit 61 in association with the user ID.

### Configuration of Evaluation Unit

The evaluation unit 68 evaluates the motor function level of the user according to the test result of each motion test determined by the success/failure determination unit 67. Specifically, the evaluation unit 68 specifies the motor function levels of the user as follows, according to the following test results.

If the test result for the user is the motion test on the left foot and on the right foot with the height dimension H of 10 cm, the evaluation unit 68 specifies the motor function level of the user as “8”.

If the test result for the user is the motion test on the left foot and on the right foot with the height dimension H of 20 cm, the evaluation unit 68 specifies the motor function level of the user as “7”.

If the test result for the user is the motion test on the left foot and on the right foot with the height dimension H of 30 cm, the evaluation unit 68 specifies the motor function level of the user as “6”.

If the test result for the user is the motion test on the left foot and on the right foot with the height dimension H of 40 cm, the evaluation unit 68 specifies the motor function level of the user as “5”.

If the test result for the user is the motion test on both feet with the height dimension H of 10 cm, the evaluation unit 68 specifies the motor function level of the user as “4”.

If the test result for the user is the motion test on both feet with the height dimension H of 20 cm, the evaluation unit 68 specifies the motor function level of the user as “3”.

If the test result for the user is the motion test on both feet with the height dimension H of 30 cm, the evaluation unit 68 specifies the motor function level of the user as “2”.

If the test result for the user is the motion test on both feet with the height dimension H of 40 cm, the evaluation unit 68 specifies the motor function level of the user as “1”.

If the test result for the user shows that the user does not succeed in any motion test, the evaluation unit 68 specifies the motor function level of the user as “0”.

The evaluation unit 68 also compares the determined motor function level with the average value of the motor function level for the user's age group and gender, and determines whether the user's state corresponds to locomotive syndrome or not. In this case, if the motor function level of the user is equal to or above the average value, the evaluation unit 68 determines that the user's state does not correspond to locomotive syndrome, whereas if the motor function level of the user is below the average value, the evaluation unit 68 determines that the user's state corresponds to locomotive syndrome. The evaluation result by the evaluation unit 68 is included in the evaluation result screen RS (see FIG. 10) generated by the presentation information output unit 65.

### Configurations of Transition Prediction Unit, Exercise Specifying Unit, and Change Estimation Unit

The transition prediction unit 69 predicts the transition of the motor function level for the coming years (for example, ten years) on the basis of the motor function level evaluated in the past by the evaluation unit 68. The transition prediction of the motor function level by the transition prediction unit 69 is carried out on the basis of statistical information stored in the storage unit 61.

The exercise specifying unit 70 specifies a type of exercise which restrains the transition (fall) of the motor function level predicted by the transition prediction unit 69, on the basis of the statistical information and the type of exercise corresponding to the motor function level, which are each stored in the storage unit 61.

The change estimation unit 71 estimates how the motor function level will change over the coming years (for example, ten years) if the user carries out the exercise of the type specified by the exercise specifying unit 70. The estimation by the change estimation unit 71 is also carried out on the basis of the statistical information stored in the storage unit 61.

### Configuration of Transmitting Unit

The transmitting unit 72 transmits various kinds of information stored in the storage unit 61 to the server 9 via the communication unit 62 and the relay device 8. The transmitting unit 72 transmits the user information and evaluation result information about the result of the motion test, for example.

The evaluation result information includes the user ID and the building BL with the motion test is carried out, the time and date of the motion test, the evaluation result by the evaluation unit 68 (motor function level), the prediction result by the transition prediction unit 69, the specification result by the exercise specifying unit 70, and the estimation result by the change estimation unit 71, or the like.

The transmitting unit 72 also transmits the evaluation result information stored in the storage unit 61 in association with the user ID, to the information processing device TE associated with the user ID (information processing device TE used by the user).

### Configuration of Relay Device

Back to FIG. 1, the relay device 8 is arranged in each building BL. The relay device 8 communicates with the control device 6 and also communicates with the server 9 via the network NT. The relay device 8 is made up of a router, for example, and is configured to be connectable for communication with the server 9 and also the information processing device TE, thus forming a LAN (local area network) inside the building BL. If the relay device 8 is made up of a wireless LAN router, the relay device 8 can wirelessly communicate with the control device 6 and the information processing device TE.

### Configuration of Information Processing Device

The information processing device TE is an external device which is configured to be able to communicate with the control device 6 via the relay device 8 and which transmits and receives information to and from the control device 6. The information processing device TE is made up of a smartphone (multifunctional mobile phone), PC (personal computer) or the like, and includes a communication unit, a control unit, a storage unit, and a display unit, though not illustrated.

The information processing device TE displays, for example, a screen similar to the evaluation result screen RS (see FIG. 10), described later, on the basis of the evaluation result information received from the control device 6. Thus, when an exercise that can be carried out outside the toilet TL is presented as the type of exercise that should be carried out by the user, the user can carry out the exercise, checking this screen.

If the information processing device TE is connected to the server 9 via the network NT, for example, using a public communication network or the like and without using the relay device 8, the information processing device TE can acquire the evaluation result information from the server 9 and can display the screen.

### Configuration of Server

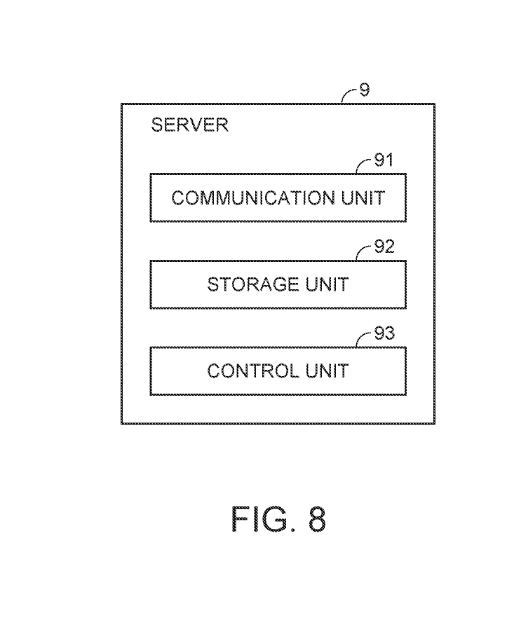

FIG. 8 is a block diagram showing the configuration of the server 9.

The server 9 is an external device which is connected for communication with the relay device 8 of each motor function evaluation device 2 via the network NT and which receives and stores information transmitted from the control device 6 via the relay device 8. The server 9 includes a communication unit 91, a storage unit 92, and a control unit 93, as shown in FIG. 8.

Of these, the communication unit 91 is a communication module which communicates with each motor function evaluation device 2 under the control of the control unit 93.

The storage unit 92 is made up of a storage such as an HDD (hard disk drive) and stores programs and data that are necessary for the operation of the server 9. As such programs, the storage unit 92 includes an OS (operating system), a management program for managing a database, described later, and a sum-up program for summing up information registered in the database. Also, as such data, the storage unit 92 stores statistical information about the determination results of the motion test for each age group and for each gender.

The database stored in the storage unit 92 includes a user table and an evaluation result table, though not illustrated.

The user table is a table in which each item of the user information is registered.

The evaluation result table is a table in which each item of the evaluation result information is registered. The time and date when the motion test is carried out, included in the evaluation result information, may be the time and date of determination when the motor function of the user is determined, or may be the time and date of reception when the evaluation result information is received.

The control unit 93 has a processing circuit such as a CPU, and reads, executes, and processes the programs and data stored in the storage unit 92. For example, when registration information of a user is newly received by the communication unit 91 from the motor function evaluation device 2, the control unit 93 associates the user ID and building ID indicated by the registration information and also registers the personal information (name, age, gender, body weight) included in the received registration information, in the user table.

Also, when the evaluation result information is received by the communication unit 91 from the motor function evaluation device 2 (control device 6), the control unit 93 registers (adds) the information included in the evaluation result information to the evaluation result table.

Moreover, when a sum-up request signal is received from the motor function evaluation device 2, the control unit 93 sums up the information registered in the evaluation result table according to the classification condition (user ID, building BL, age group and gender, or the like) included in the sum-up request signal, and sends back the sum-up result.

Also, when an information request signal which requests information stored in the storage unit 92 is received from the motor function evaluation device 2, the control unit transmits the corresponding information to the motor function evaluation device 2. For example, such information may be the statistical information.

### Motor Function Evaluation Processing

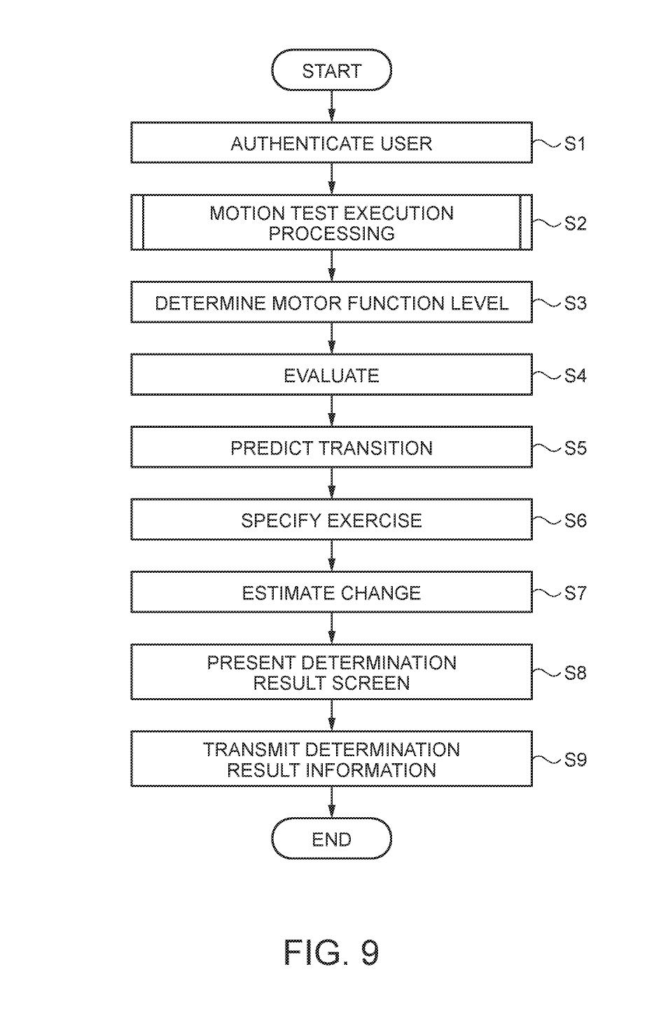

FIG. 9 is a flowchart showing motor function evaluation processing.

In the motor function evaluation system 1 described above, the motor function evaluation device 2 executes the following motor function evaluation processing. The motor function evaluation processing is carried out as the CPU forming the control device 6 executes the motor function evaluation program stored in the storage unit 61.

In the motor function evaluation processing, first the user specifying unit 64 specifies the executioner on the basis of the content of an operation signal acquired by the information acquisition unit 63 (Step S1), as shown in FIG. 9.

Next, motion test execution processing S2 is executed by the information acquisition unit 63, the presentation information output unit 65, the lift control unit 66, and the success/failure determination unit 67.

In the motion test execution processing S2, the relative height dimension H between the placement surface PL and the seat surface BS is adjusted by the lift mechanism 3 controlled by the lift control unit 66 (adjustment of height), and the height dimension H after the adjustment is acquired (acquisition of height), as described above. Also, details of the motion test are reported by the reporting unit 50 on the basis of a signal outputted from the presentation information output unit 65 (reporting). Then, the detection result of change in the pressure on the placement surface PL corresponding to the movement of the user is acquired by the information acquisition unit 63 (acquisition of change), and the success/failure of the motion test is determined by the success/failure determination unit 67 (determination of success/failure). Subsequently, details of the next motion test are decided on the basis of the success/failure of the motion test, and the relative height dimension H between the placement surface PL and the seat surface BS is adjusted by the lift mechanism 3 controlled by the lift control unit 66, on the basis of the details of this next motion test. The details of the motion test may be reported before the adjustment of the height dimension H. That is, one of the adjustment of the height dimension H and the reporting of the details of the motion test may be carried out before the other, or these may be carried out simultaneously.

As the result of the motion test execution processing, the success/failure determination unit 67 specifies the test result and the evaluation unit 68 evaluates the motor function level (Step S3).

Moreover, the evaluation unit 68 evaluates the motor function of the user on the basis of the determined motor function level (Step S4). Specifically, the evaluation unit 68 refers to the statistical information corresponding to the user's age group and gender on the basis of the age included in the user information of the user specified in Step S1, and determines whether the determined motor function level is equal to or above the average value for this age group and gender, or not. If the motor function level is equal to or above the average value, the evaluation unit 68 evaluates that the current state of the user's motor organs does not correspond to locomotive syndrome, whereas if the motor function level is below the average value, the state of the motor organs corresponds to locomotive syndrome. This Step S4 is equivalent to the evaluation of the motor function of the user.

Also, the transition prediction unit 69 predicts the transition of the motor function level for the coming years on the basis of the past determination result of the motor function level stored in the storage unit 61 (Step S5).

Moreover, the exercise specifying unit 70 specifies the type of exercise corresponding to the determined motor function level, referring to the storage unit 61 (Step S6).

In addition, the change estimation unit 71 estimates the change in the motor function level over the coming years in the case where the exercise specified by the exercise specifying unit 70 is carried out (Step S7).

The order of these Steps S5 to S7 is changeable. The processing of Step S5 may be executed after the processing of Step S6 and S7 is executed. Also, the processing of Step S5 and the processing of Steps S6 and S7 may be executed simultaneously.

After these steps, the presentation information output unit 65 generates an evaluation result screen RS (see FIG. 10) showing the processing result of Steps S3 to S7, outputs an image signal corresponding to the screen RS, and causes the reporting unit 50 (display section 51) to display the screen RS (Step S8).

Also, the transmitting unit 72 transmits the evaluation result information to the server 9 via the relay device 8 and the network NT (Step S9).

The motor function evaluation processing thus ends. In this case, if the height position of the placement surface PL does not coincide with the floor FL, the lift device 32 is driven by the lift control unit 66, thus lowering the moving unit 31. Thus, the height position of the placement surface PL coincides with the floor FL.

### Configuration of Evaluation Result Screen

FIG. 10 shows an example of the evaluation result screen RS. Here, FIG. 10 shows an example of the evaluation result screen RS presented when a 65-year-old male user who has been carrying out the motion test over the past ten years carries out the motion test anew.

The evaluation result screen RS is a screen showing the evaluation result of the user's motor function based on the test result of the motion test, and is outputted by the presentation information output unit 65, as described above. The evaluation result screen RS includes a graph display area GA arranged at the top of the screen, and an evaluation display area EA arranged at the bottom of the screen, as shown in FIG. 10.

In the graph display area GA, graphs GA1 to GA3 are arranged in series from the top to the bottom of the screen, and a legend GA4 to the respective graphs GA1 to GA3 is arranged as well.

The graph GA1 is a graph showing the result of the comparison between the executioner of the motion test and a cohort of the same age group and same gender as the executioner. The graph GA1 is a 100% stacked bar chart based on statistical information corresponding to the age and gender of the executioner. That is, the graph GA1 is a graph showing the proportion of people corresponding to each motor function level, where the entire cohort forms 100%. For example, if the executioner is a 65-year-old male user, a graph GA1 showing the determination result of the motion test with a cohort of 60 to 69 year-old male subjects is disposed, as shown in FIG. 10. Such a graph GA1 is generated by the presentation information output unit 65 functioning as the result reporting unit, on the basis of the motor function level of the executioner stored in the storage unit 61 as the determination result of the motion test, and the statistical information stored in the storage unit 61.

In this embodiment, a marker MK1 indicating the average value of the motor function level among the cohort, and a marker MK2 indicating the motor function level of the executioner are set in the graph GA1. The shapes of these markers MK1 and MK2 may be changed suitably.

The graph GA2 is a line graph showing the transition of the determination result of the motion test for the executioner over the most recent years. The graph GA2 is a graph showing the transition of the motor function level of the executioner. In this embodiment, the period covered by the graph GA2 is two years. However, this is not limiting and the period may be six months or one year and can be changed suitably. Such a graph GA2 is generated by the presentation information output unit 65 functioning as the transition reporting unit, on the basis of the motor function level stored in the storage unit 61 every time the motion test is carried out.

The graph GA3 is a line graph showing the transition of the motor function level of the executioner, the transition of the motor function level predicted for the future, and the transition of the motor function level estimated for the future in the case where the executioner carries out an exercise specified by the exercise specifying unit 70. In the example of the graph GA3 shown in FIG. 10, the transition of the motor function level of the executioner is shown by a solid line, the predicted transition is shown by a dotted line, and the estimated transition is shown by a chain dotted line. Such a graph GA3 is generated by the presentation information output unit 65 functioning as the prediction result reporting unit and the estimated state reporting unit, on the basis of the motor function level of the executioner stored in the storage unit 61, the prediction result by the transition prediction unit 69, and the estimation result by the change estimation unit 71.

In the evaluation display area EA, a level setting section EA1 where the current motor function level of the executioner is set, an exercise type setting section EA2 where a recommended type of exercise is set, and a brief comment setting section EA3 where a brief comment is set, are arranged.

In the level setting section EA1, the motor function level based on the test result of the most recent motion test is set.

In the exercise type setting section EA2, the type of exercise specified by the exercise specifying unit 70 on the basis of the test result (recommended type of exercise) is set.

In the brief comment setting section EA3, a comment for the executioner is set on the basis of the evaluation result by the evaluation unit 68 based on the test result, the transition of the motor function level of the executioner, the prediction result by the transition prediction unit 69, and the estimation result by the change estimation unit 71. This comment includes whether the state of the executioner corresponds to locomotive syndrome or not, evaluated by the evaluation unit 68, for example.

As such an evaluation result screen RS is displayed in the display section 51 in the above Step S8, the user can grasp his/her own state of motor function.

The evaluation result screen RS can also be generated at the server 9. Therefore, the information processing device TE can acquire and display the evaluation result screen RS not only by accessing the control device 6 but also by accessing the server 9, as described above.

### Advantageous Effects of Embodiment

The motor function evaluation system 1 according to the embodiment described above has the following advantageous effects.

As the lift device 32 of the lift mechanism 3 controlled by the lift control unit 66 moves the moving unit 31 up and down, the relative height dimension H between the seat surface BS of the toilet bowl TB and the placement surface PL where the user's soles are placed is adjusted. Then, details of a motion test are reported by the reporting unit 50 to which an image signal and an audio signal are inputted from the presentation information output unit 65. On the basis of the detection result of the pressure applied by the soles on the placement surface PL at the time of carrying out the motion test, the evaluation unit 68 evaluates the motor function of the user. With this configuration, the motion test can be carried out in the toilet TL. Thus, since the motion test can be carried out in the toilet TL, which is frequently used in everyday living, the motor function of the user can be determined relatively easily.

The evaluation result of the motor function by the evaluation unit 68 can be visually confirmed on the evaluation result screen RS, which is generated by the presentation information output unit 65 functioning as the result reporting unit and reported by the reporting unit 50. With this configuration, the user and other people such as medical practitioners can grasp the motor function of the user. Thus, measures corresponding to the motor function of the user can be implemented.

The evaluation result screen RS generated by the presentation information output unit 65 includes the result of the comparison between the motor function level of the user evaluated by the evaluation unit 68 and the average motor function level for the same age group and same gender as the user. Thus, by checking the result of the comparison, the user can more easily grasp whether the user's own motor function is superior or inferior to that of the same age group and same gender.

Here, in the case of adjusting the height position of the seat surface BS of the toilet bowl TB, adjusting the height position of the toilet bowl TB itself is conceivable. However, such a configuration complicates the structure and design, by needing changes to the piping for the toilet bowl TB, or the like.

In contrast, the lift mechanism 3 changes the height position of the placement surface PL, thereby changing the relative height dimension H between the placement surface PL and the seat surface BS. Thus, the height dimension H can be adjusted without significantly changing the design of the toilet TL, and the configuration of the lift mechanism 3 can be simplified.

The success/failure determination unit 67 determines the success/failure of the motion test on the basis of changes with time in each of the load of the user and the gravity center position acquired from the detection result by the first pressure detection unit 41. Then, the evaluation unit 68 specifies the motor function level indicating the motor function of the user on the basis of the determination result by the success/failure determination unit 67. Thus, the success/failure of the motion test can be properly determined by the success/failure determination unit 67, and the motor function of the user can be properly evaluated.

Here, in the determination test for locomotive syndrome, standing up by using a bounce is not regarded as a success. Therefore, the success/failure determination unit 67 can properly determine the success/failure of the motion test by determining whether the movement of the user at the time of succeeding in the motion test is by using a bounce or not. If the movement is determined as using a bounce, a notice is reported by the presentation information output unit 65 and the reporting unit 50 and therefore the user carrying out the motion test can carry out the motion test again, paying attention not to use a bounce. Thus, the user can be made to carry out the motion test properly.

The evaluation result by the evaluation unit 68 is stored in the storage unit 61, and the transition of the evaluation result is included in the evaluation result screen RS, which is generated and outputted by the presentation information output unit 65 and reported by the reporting unit 50. Thus, by checking the transition of the evaluation result, the user can securely grasp changes in his/her own motor function.

The transition prediction unit 69 predicts the future transition of the motor function of the user on the basis of the transition of the evaluation result by the evaluation unit 68, and the prediction result by the transition prediction unit 69 is included in the evaluation result screen RS reported by the reporting unit 50. Thus, if the predicted future transition of the motor function is not good, the user can be given a sense of urgency and prompted to make improvements. Meanwhile, if the transition is good, the user can be given a sense of relief.

The exercise specifying unit 70 specifies the type of exercise that should be carried out by the user, on the basis of the evaluation result by the evaluation unit 68, and this type of exercise (details) is included in the evaluation result screen RS reported by the reporting unit 50. Thus, details of a suitable exercise can be reported to the user. By carrying out this exercise, the user can restrain a reduction in his/her motor function or can improve his/her motor function.

The change estimation unit 71 estimates changes in the motor function in the case where the user carries out the exercise of the type specified by the exercise specifying unit 70, and the estimated changes in the motor function are included in the evaluation result screen RS reported by the reporting unit 50. Thus, the user can be motivated to carry out the exercise and can be prompted to carry out the exercise.

The transmitting unit 72 transmits the evaluation result information based on the result of the motion test, to an external device such as the server 9 and the information processing device TE. Thus, the evaluation result information can be accumulated in the external device. Therefore, the evaluation result can be statistically processed by the external device, for example. Moreover, since the external device acquires the evaluation result information, the user can check the determination result even when the user is not in the toilet, and therefore the convenience of the motor function evaluation system can be improved.

### Modifications of Embodiment

The invention is not limited to the embodiment and includes modifications, improvements and the like within a range that can achieve the object of the invention.

In the embodiment, the test result for the user is determined on the basis of the detection results by the respective pressure detection units 41, 42 made up of pressure sensors. However, the invention is not limited to this example. For example, an image pickup unit which picks up an image of the user's body may be provided instead of or in addition to the pressure sensors, and a dynamic image showing the movement of the user acquired by the image pickup unit may be analyzed using a technique such as an optical flow method, thus determining the test result for the user.

In the embodiment, the evaluation result screen RS including the evaluation result by the evaluation unit 68, the prediction result by the transition prediction unit 69, the type of exercise specified by the exercise specifying unit 70, and the estimation result by the change estimation unit 71, is generated by the presentation information output unit 65 and reported by the reporting unit 50 (display section 51 in particular). However, the invention is not limited to this example. That is, the processing results by these functional units 68 to 71 may be reported respectively on separate screens, or may be simply stored in the storage unit 61 or simply transmitted outside by the transmitting unit 72, without being reported to the user. Moreover, the control device 6 may be configured without having the transition prediction unit 69, the exercise specifying unit 70, and the change estimation unit 71.

In the embodiment, the evaluation unit 68 compares the motor function level of the user with the average value of the motor function level for the same age group and same gender as the user, and thus determines whether the user's state corresponds to locomotive syndrome or not. The determination result by the evaluation unit 68 is included in the evaluation result screen RS. However, the invention is not limited to this example. For example, the evaluation unit 68 need not necessarily carry out such comparison. Even in the case of carrying out comparison, a cohort may be set according to other classification conditions, such as by each age group, by each gender, or by each building BL, and the motor function level of the user may be compared with the average value of the motor function level for the cohort.

In the embodiment, the lift mechanism 3 and the lift control unit 66, forming the height changing unit and the height changing device, respectively, change the relative height dimension H between the placement surface PL and the seat surface BS by causing the lift device 32 to move up and down the moving unit 31 having the placement surface PL. However, the invention is not limited to this example. For example, the height position of the toilet bowl TB may be changed. That is, the height changing unit and the height changing device may have any configurations as long as the height dimension H can be changed and adjusted.

The motor function evaluation device 2 forming the motor function evaluation system 1 is configured using the floor FL and the toilet bowl TB in the toilet TL. However, the invention is not limited to this example. For example, a chair in which the relative height dimension between the floor and the seat surface can be changed may be used instead of the toilet bowl TB. In this case, as this chair, a chair with massage functions such as shoulder massage may be employed, for example. By providing such a chair with a movable floor part where the user's feet (soles) can be placed, and moving this movable floor part, it is possible to adjust the relative height dimension between the seat surface and the floor.

In the embodiment, the success/failure determination unit 67 determines the success/failure of the motion test on the basis of changes with time in the load of the user and the gravity center position acquired from the detection result by the first pressure detection unit 41. However, the invention is not limited to this example. That is, the success/failure determination unit 67 may determine the success/failure of the motion test by referring to other parameters.

In the embodiment, the success/failure determination unit 67 not only determines the success/failure of the motion test, but also determines whether the user stands up using a bounce or not, on the basis of the detection result by the second pressure detection unit 42, and a notice is reported if it is determined that the user uses a bounce. However, the invention is not limited to this example. That is, such determination and reporting need not necessarily be carried out.

Also, the success/failure determination unit 67 may further determine whether the soles, from the toes to the heels, are in contact with the placement surface PL on the basis of the detection result by the first pressure detection unit 41, for example.

If the toilet seat has a warm water washing function, a pressure sensor for determining the user's sitting down, provided in the toilet seat, may be used as the second pressure detection unit 42.

In the embodiment, the transmitting unit 72 transmits the evaluation result information to external devices such as the server 9 and the information processing device TE. However, the invention is not limited to this example. The transmitting unit 72 may be omitted. That is, in the invention, the relay device 8, the server 9, and the information processing device TE are not essential elements.

In the embodiment, the motor function evaluation system 1 (motor function evaluation device 2) causes the user to carry out a motion test similar to the locomotive syndrome determination test, in the toilet TL, and thus evaluates the motor function of the user. In addition to this, a configuration that enables urine test, blood pressure measurement, body weight measurement, body fat measurement, and muscle volume measurement in the toilet TL may be provided. For example, in the case of measuring the muscle volume, a configuration in which an electrode of a muscle volume sensor is arranged at a site of the toilet seat TS that directly contacts the user's skin, and in which a weak current is conducted to the user via the electrode, so that the muscle volume of the user is detected on the basis of the resistance corresponding to the value of the current, may be given as an example. The same applies to the case where the motor function evaluation device 2 is configured with the chair instead of the toilet bowl TB.

In the embodiment, details of a motion test are reported by the reporting unit 50. However, the invention is not limited to this example. That is, if the user knows the details of the motion test, the details of the motion test need not be reported. Also, the moving up and down of the placement surface PL by the lift mechanism 3 need not necessarily be carried out automatically. The placement surface PL may be moved up and down in response to an operation by the user to the operation section 53. Moreover, while the configuration in which the control device 6 determines the success/failure of the motion test on the basis of the detection result by the detection device 4 is described above, the user may determine the success/failure and input the success/failure to the operation section 53.

In the embodiment, the transmitting unit 72 transmits the evaluation result information to the server 9 located on the network NT. The server 9 may be provided in a sport facility such as a fitness club, or in a medical institution such as a hospital.

The frequency of the motion test need not be daily and may be once a month or the like. However, if the motion test is carried out every day, the motion test itself serves as training and the user can casually carry out the exercise in everyday living.

In the embodiment, when the evaluation unit 68 evaluates the motor function of the user, the motor function is quantified as the motor function levels of 0 to 8. However, the invention is not limited to this example. That is, the motor function levels for quantification may be fewer or more than the motor function levels described in the embodiment. For example, the evaluation unit 68 may set the motor function levels as follows.

If the test result for the user is the motion test on the left foot and on the right foot with the height dimension H of 20 cm, the motor function level of the user is specified as “5”.

If the test result for the user is the motion test on the left foot and on the right foot with the height dimension H of 30 cm, the motor function level of the user is specified as “4”.

If the test result for the user is the motion test on the left foot and on the right foot with the height dimension H of 40 cm, the motor function level of the user is specified as “3”.

If the test result for the user is the motion test on both feet with the height dimension H of 10 cm, the motor function level of the user is specified as “2”.

If the test result for the user is the motion test on both feet with the height dimension H of 20 cm, the motor function level of the user is specified as “1”.

If the test result for the user is the motion test on both feet with the height dimension H of 30 cm or 40 cm, or if the user fails in the motion test on both feet with the height dimension H of 40 cm, the motor function level of the user is specified as “0”.

Also, the interval of the height dimension H can be suitably changed.

In the embodiment, the control device 6 controls the operations of the lift mechanism 3, the detection device 4, and the operation panel 5 to execute the motor function evaluation processing, and communicates with the server 9 and the information processing device TE via the relay device 8. However, the invention is not limited to this example. For example, the information processing device TE may execute at least a part of the motor function evaluation processing by executing the motor function evaluation program or the like, and thus may control the operations of the lift mechanism 3, the detection device 4, and the operation panel 5 to evaluate the motor function of the user. In this case, the information processing device TE may communicate with the lift mechanism 3, the detection device 4, and the operation panel 5 via the relay device 8 and the control device 6. Also, if the lift mechanism 3, the detection device 4, and the operation panel 5 can communicate with the relay device 8 without using the control device 6, the information processing device TE may communicate with the lift mechanism 3, the detection device 4, and the operation panel 5 via the relay device 8. Moreover, the information processing device TE may communicate with the server 9 via the relay device 8 or the like. That is, the information processing device TE may implement the functions of the control device 6. The information processing device TE may acquire the height dimension H and the detection result by the detection device 4 and execute the evaluation based on these. In this case, the information processing device TE may report details of the motion test and may generate and display the evaluation result screen RS. In such a case, the information processing device TE functions as the evaluation device.

In the embodiment, the motor function evaluation program which causes the motor function evaluation device 2 (control device 6) to execute at least a part of the motor function evaluation processing is stored in the storage unit 61. However, the invention is not limited to this example. For example, at the time of executing the motor function evaluation processing, at least a part of the motor function evaluation program may be distributed from an external device such as the server 9 and executed, or may be acquired from the external device and stored in the storage unit 61. Also, at least apart of the program may be recorded in a recording medium such as a disk-type recording medium or semiconductor storage device and read out from the recording medium when the motor function evaluation processing is executed.

What is claimed is:

1. A motor function evaluation system comprising:
a height changing unit which changes a relative height dimension between a seat surface where a user sits and a placement surface where the user's soles are placed;
a first pressure detection unit which detects a pressure applied by the soles;
a movement details reporting unit which reports details of a motion test that should be carried out by the user; and
an evaluation unit which evaluates a motor function of the user on the basis of a detection result by the first pressure detection unit when the motion test is carried out by the user.

2. The motor function evaluation system according to claim 1, further comprising
a result reporting unit which reports an evaluation result of the motor function by the evaluation unit.

3. The motor function evaluation system according to claim 2, wherein
the result reporting unit reports a comparison result between a motor function level based on the detection result by the first pressure detection unit and the motor function level that is an average among at least either those of the same age group as the user or those of the same gender as the user.

4. The motor function evaluation system according to claim 1, wherein
the height changing unit is configured to be able to change the relative height dimension by changing a height position of the placement surface.

5. The motor function evaluation system according to claim 1, further comprising
a success/failure determination unit which determines a success/failure of the motion test on the basis of a change in gravity center position of the user acquired from the detection result by the first pressure detection unit,
wherein the evaluation unit evaluates the motor function of the user on the basis of a determination result by the success/failure determination unit.

6. The motor function evaluation system according to claim 5, further comprising:
a second pressure detection unit which detects the user's pressure to the seat surface; and
a notice reporting unit which reports a notice to the user,
wherein the success/failure determination unit determines whether a movement carried out by the user is a movement using a bounce or not, on the basis of a detection result by the second pressure detection unit, and causes the notice reporting unit to report a notice if the movement is determined as a movement using a bounce.

7. The motor function evaluation system according to claim 1, further comprising:
a storage unit which stores the evaluation result by the evaluation unit; and
a transition reporting unit which reports a transition of the evaluation result by the evaluation unit.

8. The motor function evaluation system according to claim 1, further comprising:
a transition prediction unit which predicts a future transition of the motor function of the user on the basis of the transition of the evaluation result by the evaluation unit; and
a prediction result reporting unit which reports a prediction result by the transition prediction unit.

9. The motor function evaluation system according to claim 1, further comprising
an exercise reporting unit which reports details of an exercise that should be carried out by the user, on the basis of the evaluation result by the evaluation unit.

10. The motor function evaluation system according to claim 9, further comprising:
a change estimation unit which estimates a change in the motor function in the case where the user carries out the exercise; and
an estimated state reporting unit which reports the estimated change in the motor function of the user.

11. The motor function evaluation system according to claim 1, further comprising
a transmitting unit which transmits information based on the evaluation result by the evaluation unit to an external device.

12. A motor function evaluation method comprising:
adjusting a relative height dimension between a seat surface where a user sits and a placement surface where the user's soles are placed;
reporting details of a motion test that should be carried out by the user; and
evaluating a motor function of the user on the basis of a change in a pressure applied to the placement surface by the user's soles when the motion test is carried out by the user.

13. A motor function evaluation program executed by an evaluation device which evaluates a motor function of a user, the program causing the evaluation device to execute:
causing a height changing device which changes a relative height dimension between a seat surface where a user sits and a placement surface where the user's soles are placed, to adjust the height dimension;
causing a reporting device to report details of a motion test that should be carried out by the user; and
evaluating a motor function of the user on the basis of a detection result by a pressure detection device which detects a change in a pressure applied to the placement surface by the user's soles when the motion test is carried out by the user.

14. An evaluation device comprising:
a height acquisition unit which acquires a relative height dimension between a seat surface where a user sits and a placement surface where the user's soles are placed;
a change acquisition unit which acquires a change in a pressure applied to the placement surface by the user's soles when a motion test is carried out by the user; and
an evaluation unit which evaluates a motor function of the user on the basis of the height dimension and the change in the pressure.

15. A motor function evaluation program executed by an evaluation device which evaluates a motor function of a user, the program causing the evaluation device to execute:
acquiring a relative height dimension between a seat surface where a user sits and a placement surface where the user's soles are placed;
acquiring a change in a pressure applied to the placement surface by the user's soles when a motion test is carried out by the user; and
evaluating the motor function of the user on the basis of the height dimension and the change in the pressure.
